(19) United States
(12) Patent Application Publication
(10) Pub. No.: US 2024/0390273 A1
(43) Pub. Date: Nov. 28, 2024

(54) Pharmaceutical Composition for Dry Powder Inhalation and Preparation Method Thereof

(71) Applicant: ASG INSPIRATION LABORATORY (SINGAPORE) PTE. LTD., Singapore (SG)

(72) Inventors: Sung Tsai Yu, Taipei City (TW); Hao-Wei Huang, Taipei City (TW); Johnson Lee, Taipei City (TW); Hsin-Ke Li, Taipei City (TW); Jao Juen Hung, Taipei City (TW); Shih Pan Chao, Taipei City (TW); Wen-Hui Fu, Taipei City (TW)

(21) Appl. No.: 18/671,722

(22) Filed: May 22, 2024

Related U.S. Application Data
(60) Provisional application No. 63/504,197, filed on May 24, 2023.

(51) Int. Cl. A61K 9/00; A61K 9/16; A61K 31/47; B82Y 5/00

(52) U.S. Cl. CPC A61K 9/0075; A61K 9/1623; A61K 9/1647; A61K 9/1652; A61K 31/47; B82Y 5/00

(57) ABSTRACT

A pharmaceutical composition for dry powder inhalation includes an active ingredient and a first pharmacologically acceptable excipient. The active ingredient includes bedaquiline or a pharmaceutically acceptable salt thereof. The first pharmacologically acceptable excipient includes amino acid, polysaccharide, phospholipid, polylactic acid, polylactic acid copolymer, or a combination thereof. A method of preparing a pharmaceutical composition for dry powder inhalation is also provided.

### TECHNICAL FIELD

Anti-tuberculosis drug pharmaceutical preparation of bedaquiline includes a bedaquiline solid composition and an excipient. The bedaquiline solid composition includes bedaquiline or a pharmaceutically acceptable salt thereof, amino acid, phospholipid, polylactic acid and polysaccharide. The purpose of bedaquiline pharmaceutical preparation obtained from the present disclosure is to produce the bedaquiline solid composition by using spray drying. Through spray drying, particle sizes and shapes of finely divided particles can be modified to achieve better lung distribution and therapeutic effect under a combination of specific ratios.

### BACKGROUND TECHNIQUE

Bedaquiline is a drug used to treat tuberculosis, and is traditionally administered orally.

### CONTENT OF INVENTION

The present disclosure provides a preparation method of bedaquiline pharmaceutical preparation. The purpose of which is to control particle sizes of 90% particles below 5 μm and shapes of particles are spherical-shaped form or spherical-like form, which is not irregular shapes produced by grinding, to achieve better effect of aerodynamics and enable bedaquiline pharmaceutical preparation to be easily prepared for inhalation dosage form.

In one embodiment, a bedaquiline pharmaceutical preparation includes a bedaquiline solid composition and an excipient, in which the bedaquiline solid composition includes bedaquiline or a pharmaceutically acceptable salt thereof, amino acid, phospholipid, polylactic acid and polysaccharide, and they are mixed according to a specific ratio and then prepared for fine particles with specific particle sizes by spray drying.

In one embodiment, a dose of bedaquiline pharmaceutical preparation composition includes bedaquiline or a pharmaceutically acceptable salt thereof accounting for 75˜98%, amino acid (leucine, or the like) 25˜5%, polysaccharide 20˜5%, polylactic acid 15˜2% or phospholipid 20˜5%.

In one embodiment, a preparation method of fine particles of the bedaquiline pharmaceutical preparation composition of bedaquiline or a pharmaceutically acceptable salt thereof and amino acid (such as leucine) include the steps as follows: step a: dissolving bedaquiline or a pharmaceutically acceptable salt thereof by an organic solvent and dissolving amino acid (such as leucine) by water; then, the two are mixed with each other in a specific ratio to form a solution. The optimal mixing ratio is bedaquiline:amino acid=70˜98:30˜2 (w/w), and the best ratio is 95:5 (w/w). The total concentration of the solution is 5%˜0.5%, such as 1.0 w/v %; step b: spray drying the solution obtained from step a to obtain fine particles by using a nanoparticle spray dryer. An outlet temperature of spray drying is 50˜90 degree, an ultrasonic atomization percentage is 40˜80%.

Sample Solution

| | | |
|---|---|---|
| Bedaquiline:Leucine | 95:5 | (w/w) |
| Concentration | 1.0 | w/v % |

In one embodiment, the solvent used in abovementioned step a is water, ethanol, methanol, methylene chloride, ethyl acetate, acetonitrile, or acetone.

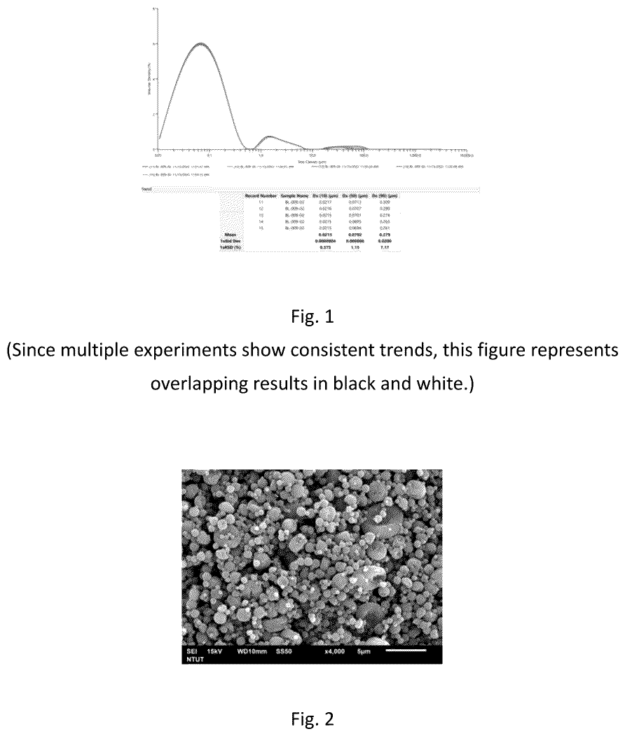

In one embodiment, particle sizes of the fine particles of the solid composition of bedaquiline and amino acid are smaller, generally <2 μm. The more optimized particle size can reach 0.1-1 μm, as shown in FIG. 1. The shape of the obtained particles is close to round, as shown in FIG. 2, which can meet the requirement of particle sizes for pulmonary drug delivery and help the fine particles to extend flight distance in lungs.

In one embodiment, a preparation method of the fine particles of the bedaquiline pharmaceutical preparation composition of bedaquiline or a pharmaceutically acceptable salt thereof and polysaccharide (such as chitosan) includes the steps as follows: step a: dissolving bedaquiline or a pharmaceutically acceptable salt thereof by an organic solvent and dissolving polysaccharide (such as chitosan) by anhydrous alcohol; then, the two are mixed with each other according to a specific ratio to form a solution, and appropriate acid is added (such as 0.1˜0.01% acetic acid) to help the solute dissolve. The optimal mixing ratio is bedaquiline:polysaccharide (such as chitosan)=80˜95:20˜5, and the best ratio is 95:5 (w/w). The total concentration of the solution is 3% ˜0.5%; step b: after filtering the solution obtained in step a with filter paper, spray drying the solution to obtain fine particles by using a nanoparticle spray dryer. An outlet temperature of spray drying is 50˜70 degree, an ultrasonic atomization percentage is 40˜70%.

Sample solution

| | | |
|---|---|---|
| Bedaquiline:Chitosan | 95:5 | (w/w) |
| Concentration | 2.0 | w/v % |

In one embodiment, polysaccharide is chitosan, a molecular weight of chitosan is MW50000˜190000, and the deacetylation acidity is above 90%. The solvent used in abovementioned step a is ethanol, methanol, or acetone.

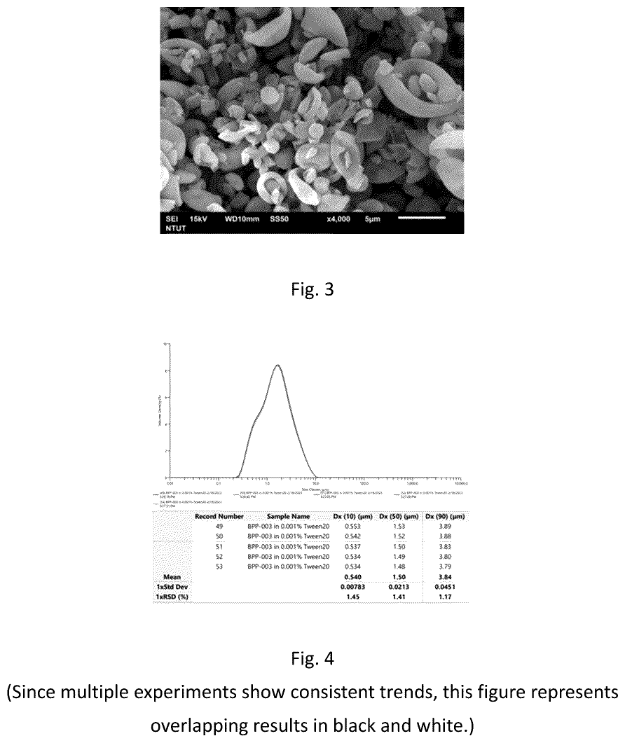

In one embodiment, particle sizes of the fine particles of the solid composition of bedaquiline and polysaccharide are smaller, generally <6 μm. The more optimized particle size can reach 3-5 μm (figures remain to be added). The shape of the obtained particles is close to round, like red blood cells, as shown in FIG. 3, which can meet the requirement of particle sizes for pulmonary drug delivery and help fine particles to extend flight distance in lungs.

In one embodiment, a preparation method of fine particles of the bedaquiline pharmaceutical preparation composition of bedaquiline or a pharmaceutically acceptable salt thereof and phospholipid (such as dipalmitoylphosphatidylcholine (DPPC) or distearoyl phosphatidyl choline (DPSC)) includes the steps as follows: step a: dissolving bedaquiline or a pharmaceutically acceptable salt thereof by an organic solvent and dissolving phospholipid (such as DPPC, DPSC) by alcohol or methanol; then, the two are mixed with each other in a specific ratio to form a solution. The optimal mixing ratio is bedaquiline:phospholipid (such as dipalmitoylphosphatidylcholine or distearoyl phosphatidyl choline)=80˜99:20˜1, and the best ratio is 98.5:1.5 (w/w). The total concentration of the solution is 3%˜0.5%; step b: spray drying the solution obtained in step a to obtain fine particles by using a nanoparticle spray dryer. An outlet temperature of spray drying is 50˜70 degree, and an ultrasonic atomization percentage is 40˜70%.

Sample Solution

| | | |
|---|---|---|
| bedaquiline:dipalmitoylphosphatidylcholine | 99:1 | (w/w) |
| Concentration | 2.0 | w/v % |
| Bedaquiline:Chitosan | 99:1 | (w/w) |

In one embodiment, phospholipid is dipalmitoylphosphatidylcholine (DPPC), and molecular weight Mw is 744, phospholipid is distearoyl phosphatidyl choline (DPSC), and molecular weight Mw is 790. The solvent used in abovementioned step a is ethanol, methanol, or acetone.

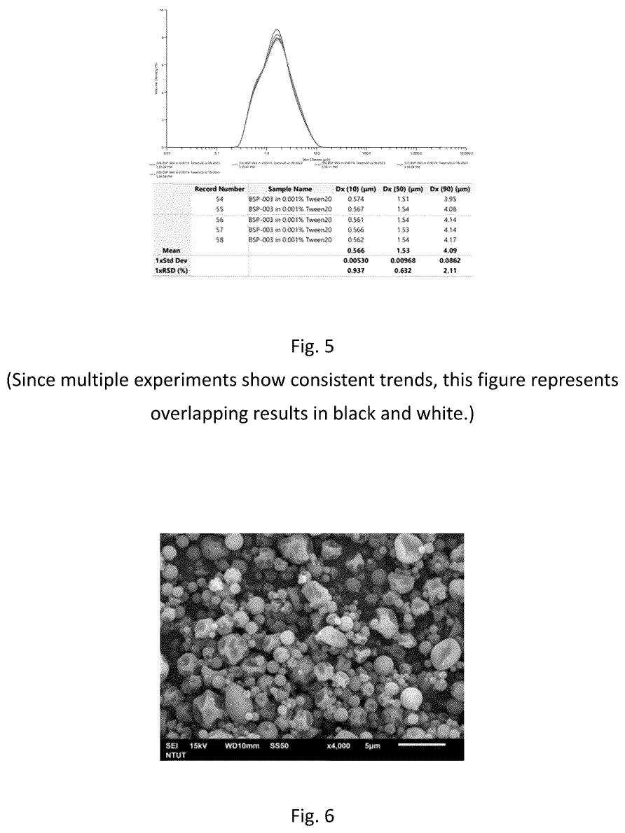

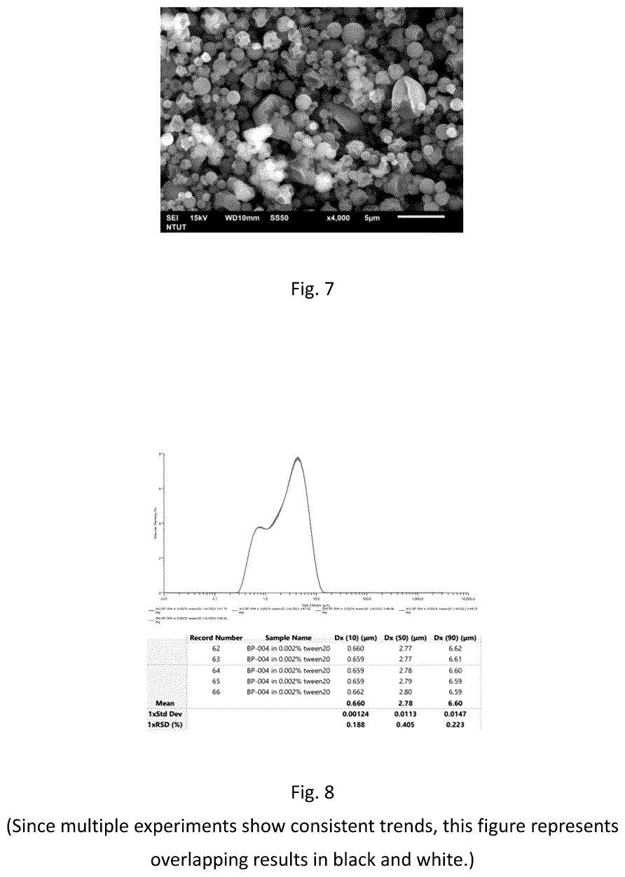

In one embodiment, particle sizes of the fine particles of the solid composition of bedaquiline and phospholipid (such as dipalmitoylphosphatidylcholine or distearoyl phosphatidyl choline) are smaller, generally <9 μm. The more optimized particle size can reach 3-5 μm, as shown in FIG. 4 and FIG. 5. The shape of the obtained particles is close to round spherical-shaped form, as shown in FIG. 6 and FIG. 7, which can meet the requirement of particle sizes for pulmonary drug delivery and help fine particles to extend flight distance in lungs.

In one embodiment, a preparation method of fine particles of the bedaquiline pharmaceutical preparation composition of bedaquiline or a pharmaceutically acceptable salt thereof and phospholipid (such as dipalmitoylphosphatidylcholine or distearoyl phosphatidyl choline) includes the steps as follows: step a: dissolving bedaquiline or a pharmaceutically acceptable salt thereof by an organic solvent and dissolving polylactic acid (such as poly (lactic-co-glycolic acid (PLGA)) by water; then, the two are mixed with each other in a specific ratio to form a solution. The optimal mixing ratio is bedaquiline:polylactic acid (such as poly (lactic-co-glycolic acid (PLGA))=90˜99:10˜1 (w/w), and the best ratio is 95:5 (w/w). The total concentration of the solution is 3%˜0.5%; step b: spray drying the solution obtained from step a to obtain fine particles by using a nanoparticle spray dryer. An outlet temperature of spray drying is 40˜50 degree, an ultrasonic atomization percentage is 40˜70%.

Sample Solution

| | | |
|---|---|---|
| Bedaquiline:Poly(lactic-co-glycolic acid | 95:5 | (w/w) |
| Concentration | 2.0 | w/v % |

In one embodiment, at least one of polylactic acid (PLA), poly (lactic-co-glycolic acid (PLGA), etc. is selected. Viscosity of PLA is 0.49˜1.92 dl/g. The solvent used in the aforementioned step a is methylene chloride, acetic acid, ethyl acetate, acetonitrile, or acetone.

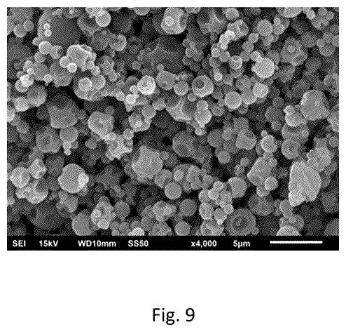

In one embodiment, particle sizes of the fine particles of the solid composition of bedaquiline and polylactic acid are smaller, generally <8 μm. The more optimized particle size can reach 3-5 μm, as shown in FIG. 8. The shape of the obtained particles is close to round spherical-shaped form, hollow spherical-shaped form or golf ball shape, as shown in FIG. 9, which can meet the requirement of particle sizes for pulmonary drug delivery and help fine particles to extend flight distance in lungs.

In one embodiment, the fine particles prepared by the abovementioned steps can be packed into dry powder inhalation device such as capsules, aluminum foil blisters or drug storage tanks for use, or the fine particles can be mixed with lactose or mannitol with appropriate particle sizes and then packed into dry powder inhalation device such as capsules, aluminum foil blisters and drug storage tanks for use.

In one embodiment, the fine particles can be mixed with lactose or mannitol with appropriate particle sizes according to appropriate ratios, such as the two tables listed below.

| | Mixing Ratio |
|---|---|
| Lactose monohydrate A | 95%~70% |
| (particle size scope: | |
| D50: 5~50 μm, D90: 20~80 μm) | |
| Lactose monohydrate B | 5%~30% |
| (particle size scope: | |
| D50: 30~125 μm, D90: 50~300 μm) | |
| Fine particles of Bedaquiline composition | 0.005%~1% |

| | Mixing Ratio |
|---|---|
| Mannitol A | 95%~70% |
| (particle size scope: | |
| D50: 5~60 μm, D90: 20~100 μm) | |
| Mannitol B | 5%~30% |
| (particle size scope: | |
| D50: 30~125 μm, D90: 50~300 μm) | |
| Fine particles of Bedaquiline composition | 0.005%~1% |

The particles are mixed according to the abovementioned ratios in a high speed mixer, in which the particles can be mixed evenly by selecting the different mixing speeds of 500 rpm˜2000 rpm according to the mixing amount. The mixture is then packed into dry powder inhalation device such as capsules, aluminum foil blisters or drug storage tanks.

In one embodiment, the fine particles can be mixed with lactose or mannitol with appropriate particle sizes, the prepared mixture product can enhance the ratio of fine particle fraction distributed in lungs and improve the therapeutic effect.

In one embodiment, the mixture product can enhance the ratio of fine particle fraction distributed in lungs. The flight effect of the powder (figures remain to be added) is evaluated by using NGI (Next generation impactor) to measure in vitro deposition of different stages. Compared with pure composition powder with particle sizes similar to which of the fine particles prepared by the abovementioned steps, a distribution figure of drug content in different stages represents that the fine particles prepared by the abovementioned steps or the fine particles mixed with lactose or mannitol with appropriate particle sizes have higher retention in the 5th, 6th and 7th stages of the NGI device, indicating that the flight distance of the powder is farther. An in vitro atomization parameter, fine particle fraction, is calculated, in which the fine particle fraction is a ratio of fine particles with particle sizes ≤5 u m in the agent relative to a total content of the drug. The higher the FPF content, the higher the proportion of drug distributed in the lungs. Compared with pure composition powder with particle sizes similar to which of the fine particles prepared by the abovementioned steps, fine particles prepared by the abovementioned steps or the fine particles mixed with lactose or mannitol with appropriate particle sizes have higher FPF.

What is claimed is:

1. A pharmaceutical composition for dry powder inhalation comprising:
an active ingredient, wherein the active ingredient comprises bedaquiline or a pharmaceutically acceptable salt thereof; and
a first pharmacologically acceptable excipient, comprising amino acid, polysaccharide, phospholipid, polylactic acid, polylactic acid copolymer, or a combination thereof.

2. The pharmaceutical composition of claim 1, wherein a weight percentage of the active ingredient is from 50% to 99% and a weight percentage of the first pharmacologically acceptable excipient is from 1% to 50% based on 100% by weight of the active ingredient and the first pharmacologically acceptable excipient.

3. The pharmaceutical composition of claim 1, wherein a weight percentage of the amino acid is from 1% to 20% based on 100% by weight of the active ingredient and the first pharmacologically acceptable excipient.

4. The pharmaceutical composition of claim 1, wherein the amino acid comprises glycine, alanine, valine, leucine, isoleucine, phenylalanine, tryptophan, tyrosine, aspartic acid, histidine, asparagine, glutamic acid, lysine, glutamine, methionine, arginine, serine, threonine, cysteine, proline or a combination thereof.

5. The pharmaceutical composition of claim 1, wherein a weight percentage of the polysaccharide is from 1% to 20% based on 100% by weight of the active ingredient and the first pharmacologically acceptable excipient.

6. The pharmaceutical composition of claim 1, wherein the polysaccharide comprises chitosan, chitosan salt or a combination thereof.

7. The pharmaceutical composition of claim 1, wherein a weight percentage of the phospholipid is from 1% to 50% based on 100% by weight of the active ingredient and the first pharmacologically acceptable excipient.

8. The pharmaceutical composition of claim 1, wherein the phospholipid comprises dipalmitoyl phosphatidylcholine, distearoyl phosphatidyl choline or a combination thereof.

9. The pharmaceutical composition of claim 1, wherein a weight percentage of the polylactic acid is from 1% to 50% based on 100% by weight of the active ingredient and the first pharmacologically acceptable excipient.

10. The pharmaceutical composition of claim 1, wherein a weight percentage of the polylactic acid copolymer is from 1% to 50% based on 100% by weight of the active ingredient and the first pharmacologically acceptable excipient.

11. The pharmaceutical composition of claim 1, wherein the polylactic acid copolymer comprises poly (lactic-co-glycolic acid).

12. The pharmaceutical composition of claim 1, wherein the active ingredient and the first pharmacologically acceptable excipient forms a finely divided particle having a particle size of from 50 nm to 6 μm.

13. The pharmaceutical composition of claim 12, wherein the finely divided particle is provided in a solid spherical-shaped form, a hollow spherical-shaped form, a solid polyhedron form or a combination thereof.

14. The pharmaceutical composition of claim 1, further comprising a second pharmacologically acceptable excipient different from the first pharmacologically acceptable excipient.

15. The pharmaceutical composition of claim 14, wherein a weight percentage of the second pharmacologically acceptable excipient is from 70% to 99.995% based on 100% by weight of the pharmaceutical composition.

16. The pharmaceutical composition of claim 14, wherein the second pharmacologically acceptable excipient comprises lactose, mannitol or a combination thereof.

17. A method of preparing a pharmaceutical composition for dry powder inhalation, comprising:
dissolving an active ingredient in a first solvent to form a first solution, wherein the active ingredient comprises bedaquiline or a pharmaceutically acceptable salt thereof;
dissolving a first pharmacologically acceptable excipient in a second solvent to form a second solution, wherein the first pharmacologically acceptable excipient comprises amino acid, polysaccharide, phospholipid, polylactic acid, polylactic acid copolymer, or a combination thereof;
mixing the first solution and the second solution to form a mixture; and
spray drying the mixture to form a finely divided particle.

18. The method of claim 17, wherein the first solvent and the second solvent comprise an organic solvent.

19. The method of claim 17, wherein a weight percentage of the active ingredient and the first pharmacologically acceptable excipient is from 0.5% to 3% based on 100% by weight of the mixture.

20. The method of claim 17, wherein a weight ratio of the active ingredient and the first pharmacologically acceptable excipient in the mixture is from 1:1 to 199:1.

21. The method of claim 17, wherein spray drying the mixture is performed at an outlet temperature of from 35° C. to 110° C.

22. The method of claim 17, wherein an ultrasonic atomization percentage of the mixture at the step of spray drying the mixture is from 25% to 85%.

23. The method of claim 17, further comprising mixing the finely divided particle with a second pharmacologically acceptable excipient different from the first pharmacologically acceptable excipient.

24. The method of claim 23, wherein the second pharmacologically acceptable excipient comprises a first size group, a second size group or a combination thereof, wherein a volume-basis particle size distribution of the first size group is different from a volume-basis particle size distribution of the second size group.

25. The method of claim 24, wherein a particle size D50 of the first size group is from 5 μm to 50 μm and a particle size D50 of the second size group is from 30 μm to 125 μm.

26. The method of claim 17, further comprising mixing the finely divided particle with a flavoring agent.
